(19) United States
(12) Patent Application Publication
(10) Pub. No.: US 2020/0000644 A1
(43) Pub. Date: Jan. 2, 2020

(54) HEAD TRAUMA BANDAGE CAP AND METHOD

(71) Applicants: Mitchell Kastros, Carmel, CA (US); Marc Etchells, Westhampton, NA (US)

(72) Inventors: Mitchell Kastros, Carmel, CA (US); Marc Etchells, Westhampton, NA (US)

(73) Assignee: First Responder Solutions, LLC, Carmel, CA (US)

(21) Appl. No.: 16/539,067

(22) Filed: Aug. 13, 2019

Related U.S. Application Data
(63) Continuation-in-part of application No. 15/590,935, filed on May 9, 2017.
Application No. 15/590,935 is a continuation-in-part of application No. 14/789,569, filed on Jul. 1, 2015, now Pat. No. 10,376,417.

(51) Int. Cl. A61F 13/12; A61F 7/02; A42B 1/04

(52) U.S. Cl. CPC A61F 13/12; A61F 2007/0008; A42B 1/043; A61F 7/02

(57) ABSTRACT

An emergency trauma stretch bandage cap shaped as a skull cap with roll able edges capable of holding hot or cold packs, which is placed on the cranium to cover the crown, forehead, back of the head, ears, sides of the head around the ears, and the temples of an injured patient with minimal movement of the neck and spine made of two or more layers of a stretchable warp knit fabric cut and sewn to form a form-fitting head bandage shape to apply sufficient pressure to suppress bleeding.

### CROSS REFERENCED RELATED APPLICATIONS

This application is a continuation-in-part patent application of the “Head Trauma Bandage Cap and Method, Ser. No. 15/590,935 filed May 9, 2017, which is a continuation-in-part patent application entitled “Head Trauma Bandage Cap and Method”, Ser. No. 14/789,569, filed Jul. 1, 2015, which is a continuation-in-part patent application entitled “Head Trauma Bandage Cap and Method”, Ser. No. 13/560,410, filed Jul. 27, 2012, which is a continuation-in-part patent application entitled “Helmet Trauma Bandage and Method”, Ser. No. 12/807,288, filed Sep. 10, 2010, which is a continuation-in-part of a continuation-in-part patent application of the continuation-in-part patent application entitled “Head Trauma Bandage and Method”, Ser. No. 12/586,431, filed Sep. 22, 2009, which is a continuation-in-part of the patent application entitled “Head Trauma Cap Bandage”, Ser. No. 12/156,512 filed Jun. 2, 2008.

### BACKGROUND OF THE INVENTION

### Field

The present invention relates to bandages and trauma treatment. In particular, it relates to a stretch emergency trauma bandage beanie capable of holding wound dressings and/or hot or cold packs and incorporated hemostatic functionality, which is placed on the cranium to cover the crown, forehead, back of the head, sides of the head around the ears, and the temples of an injured patient with minimal movement of the neck and spine.

### Description of Related Art

Various bandages are known in the art. Cummings et al, U.S. Design Pat. No. D736,937 issued Aug. 18, 2015 discloses a head bandage design with a strap to apply head wound pressure. Cummings et al, U.S. Pat. No. 9,149,393 issued Oct. 19, 2018 discloses a head bandage also using a strap system to apply head wound pressure. Cummings et al, U.S. Pat. No. 8,262,601 issued Sep. 11, 2012 discloses a head bandage with a strap system, and pockets to hold wound dressing and/or cold packs and hot compresses for head wounds.

Boukanov et al., U.S. Pat. No. 6,762,337 issued Jul. 13, 2004 discloses a multi-purpose pressure bandage for body wounds utilizing an expansion bladder, which inflates to compress an affixed bandage against an injured patient's wounds. Boukanov et al. specifically states its system design is to provide a pressure dressing. To apply pressure, the device incorporates the use of a carbon dioxide gas container with an inlet valve for inflating a bladder in the bandage on site to apply additional pressure (resistance pressure or inflating to create pressure) to the wound to control bleeding.

The Boukanov et al. embodiment for head wounds has the compression bandage shaped like a cap to secure about the head. The Boukanov et al head bandage configuration has a bladder with a coextensive gauze bandage liner and a gas cartridge hidden in a pouch at a bottom edge. Elongated straps extend diametrically from the bottom edge for securing the dome-shaped pressure bandage to a head injury. Once in place, the bladder of the bandage is inflated to apply pressure to the wound. Although the application of pressure to control bleeding is taught to be the standard when treating soft tissue injuries, it is contraindicated with regard to bleeding associated with a head injury, requiring only gentle pressure.

Boukanov et al.'s compression bandage is not suitable for head injuries. Head injuries are usually associated with intracranial swelling, which causes excess pressure on the brain and towards the skull. Pressure treatment similar to Boukanov et al. applied to a head injury through compression compounds the problem of internal pressure to the brain and skull. Through this compression method the pressure applied by treating the injury creates even more pressure on the brain, and this can lead to a rapid deterioration of the condition of the patient. In addition, long-term and permanent brain damage can occur from the application of a pressure dressing, which, in the worst case, can lead to the death of the patient.

The standard of treatment for a head injury is to apply gentle pressure for controlling bleeding, and for applying a cold pack to control intracranial swelling associated with head trauma. The idea of applying “gentle pressure” is to not exceed the amount of pressure being exerted inside the cranium resulting from head trauma. In addition, as stated in Eliyahu-Gross et al., US 2016/0121819 published May. 5, 2016,
“In the treatment of moderate to severe traumas, controlling bleeding is essential and critical to minimize blood loss. The process of healing injuries begins with the adhesion and agglutination of platelets to injured tissue, and the simultaneous liberation of thromboplastin from injured cells. To aid in this process, hemostatic wound dressings are used for sealing an injured site, thereby reducing the loss of blood, activating the clotting mechanisms and promoting hemostasis.”

The present invention discussed below is designed to be consistent with the standard for treating head injuries, and does not have any similarities with the Boukanov et al bladder compression system with regard to its application. It does not have any features, which create excess pressure, compromise cervical-spinal precautions or in any other way challenge the well-being of the patient with a head injury.

If the Boukanov et al. bladder pressure regulator fails, it also may result in significant pressure, which can cause serious head injuries where intra cranial fluids build up causing the head to swell. In addition, if improperly inflated, circulation may be cut off. The bandage also suffers from compression problems if the gas container is empty, or fails to inflate the bladder. Under these circumstances, the Boukanov et al pressure bandage may aggravate the patient's head injuries. Further, if the Boukanov et al. bladder is pierced accidentally during emergency use, an ill-fitting head wrap results.

Lundell et al., U.S. Design Pat., Des. 295,446, issued Apr. 26, 1988 is a head bandage protector that would require first conventionally wrapping the patient with bandages, which may compromise cervical spine immobilization depending upon how the bandage wraps are administered.

Fye, U.S. Pat. No. 5,031,609, issued Jul. 16, 1991 is a postoperative compression bandage for the head, which would also require conventional bandaging before compression application; again possibly compromising cervical spine immobilization.

Neither Lundell et al, nor Fye are bandages with a weather resistant cover for rapid application in the field to avoid moving the neck or spine during emergency trauma applications.

Cited for general interest are: Sherwood, U.S. Pat. No. 5,044,031, issued Sep. 3, 1991 discloses passive warming articles for traumatized individuals suffering from hypothermia, shock or exposure. Kun, U.S. Pat. No. Des. 354,376, issued Feb. 14, 1995 discloses a head-cooling cap. Hujar et al., U.S. Pat. No. 5,557,807 issued Sep. 24, 1996 discloses headwear including coolant means. Ameer, U.S. Pat. No. 6,228,041, issued May 8, 2001 discloses a lightweight portable scalp vibrating and hair growth-stimulating device. Komachak, U.S. Publication No. US2007/0074326, dated Apr. 5, 2087, discloses a headgear with cooling device formed using a woven or non-woven material. Wang, U.S. Pat. No. 4,744,106, issued May 17, 1988 discloses an engineering cap with fan device structure for ventilation of the hard hat. Augustine et al., U.S. Pat. No. 5,860,292 issued Jan. 19, 1999 discloses an inflatable thermal blanket with head covering for convectively cooling the body. Robinson et al., U.S. Pat. No. 6,678,896, issued Jan. 20, 2804 discloses a sports towel. Ronquillo, U.S. Pat. No. 5,666,668 issued Sep. 16, 1997 discloses a cap with front size adjustment and rear flap. Dixon, U.S. Pat. No. 5,960,477 issued Oct. 5, 1999 discloses a hat with folded rim and visor. Dumas et al., U.S. Pub. No. 2005/0827227 published Feb. 3, 2005 discloses a disposable water resistant cover for, medical applications. Reeves, U.S. Pat. No. 6,747,561 issued Jun. 8, 2004 discloses a bodily worn device, which provides for digital storage and retrieval of a user's medical records, drug prescriptions, medical history, organ donor instructions, and personal identification for use in an emergency or routine medical situation. Zucker et al., U.S. Publication No. US200S/0193491 published Sep. 8, 2885, discloses a pediatric emergency transport device. McKay, U.S. Pat. No. 5,305,470, issued Apr. 26, 1994 discloses a sports band. Brisbane, U.S. Pat. No. 945,839, issued Jan. 11, 1910 is a sleeping cap unsuitable for use as a bandage, and may not expand sufficiently to accommodate larger heads. The elasticized Brisbane sleeping cap using elasticized side to apply pressure for holding the cap onto the head could adversely affect intracranial pressure from a head wound and aggravate the wound tissue when slid over the head. Dixon, U.S. Pat. No. 5,960,477, issued Oct. 5, 1999, is a snow hat with folded rim requiring the head to be lifted for placement, again aggravating spinal injuries. Dumas et al. U.S. Publication 2005/0027227 published Dec. 3, 2005 is a medical disposable water resistant cover for medical applications. Shifrin, U.S. Pat. No. 5,173,970, issued Dec. 29, 1992 discloses a visored cap-type protective segmented helmet for bicyclists and the like, which can be used as a pouch.

None of the above references provides an emergency head trauma bandage cap that can be applied and placed in less than one minute, can be self-administered, used with a C-Collar, provide shorter fire and ambulance on-scene time for faster patient delivery to an ER, has a simple FDA regulatory path (non-invasive), is useful in tactical combat, multi-casualty and triage situations, and can be utilized in the first responder, emergency room, and post-surgery settings. The invention described below provides such an invention and method of using it.

### BRIEF SUMMARY OF THE INVENTION

The invention comprises a stretch emergency trauma bandage cap capable of holding wound dressings, and/or hot or cold packs, which is placed on the cranium to cover the crown, forehead, back of the head, sides of the head around the ears, and the temples of an injured patient with minimal movement of the neck and spine. It comprises a flexible head trauma bandage cap made of two or more layers of a stretchable knit fabric cut and sewn to form a form-fitting head bandage skull cap shape with rollable, liftable edges that can be adjustably rolled to apply varying pressure to head wounds. These fabrics may be knit using a variety of techniques including warp, circular or 3D knit. The knit structure provides conformity and stretch. They may have multidimensional elasticity of up to 4 ways. Lycra fiber is often used as a component in the knit to provide stretch and recovery and allows the fabric to be more durable and resistant to the corrosive action of chlorine. When made of opaque Lycra fibers, the fabric filters the harmful rays of the sun shielding the eyes of a patent when covered.

The exterior layer may be a weather resistant or waterproof exterior with periphery edges, top and sides sized to fit about and cover a forehead/crown, sides, ears, and back of a head of a patient with a head trauma with liftable edges to expose a patient's ears to enable caregivers to observe any fluid discharge from the ears.

Water repellent treatment of the exterior layer may be made by applying a thin film such as polyurethane, hydrophilic fluorocarbon or wax to provide water beading and limit moisture droplets from entering, but allow vapor transmission.

The interior layer is made of a fibrous knit structure contact surface, with enough stretch when placed on a patient to apply minimal pressure to a patient's head to control bleeding without aggravating intracranial pressure. Preferred fabrics provide balance stretch in every direction—sometimes called 4-way stretch, which provides 100% full recovery. Some fabrics can be hydrated and chilled or frozen to provide an extended duration cooling device. The filaments or fibers, blends of filaments or fibers, may be coated or treated filaments or fibers to provide hemostatic functionality of the interior layer capable of stopping the flow of blood. Hemostasis is the natural process in which blood flow slows and a clot forms to prevent blood loss through a damaged blood vessel. The opposite of hemostasis is hemorrhage. When a hemostatic agent is applied to a wound the damaged blood vessel constricts to stop the flow of blood out of the vessel. The use of hemostatic agents in wound care is critical in preventing blood loss from severe wounds in which the natural process of hemostasis is not effective for controlling blood loss.

These fibers may be formed in a discrete layer placed or laminated as the interior or may be knit into a two-sided fabric in which the hemostatic filaments or fibers are on the one side while the exterior filaments or fibers are on the other side. The two layers of fabric are cut sand sewn with seams positioned so no edges are against the patient's skin when applied.

Hemostatic activity is promoted or achieved via use of a number of materials such as:
Thrombin or certain other naturally derived proteins.
Chitosan, zeolites and many clays/minerals in powder form.
Absorbent/adhesive structures like the WoundClot™ CMC gel produced by Core Scientific under the WoundClot brand.
Fibers/filaments, generally ionic to highly ionic or with certain functional groups on the surface. Beeken Biomedical branded NuClot bandage is an example. Cotton fibers/fabrics which may be enhanced with certain cotton chemical modifications also.
Non-hemostatic fibers/filaments which have been coated or surface treated with any of the groups above.
Lastly, hemostatic fibers/filaments which have been additionally coated or treated.

The silica fibers, and all others are positively charged, which assist in the ability to accelerate clotting, wick fluids, absorb fluids and/or form a gel. The disadvantage of the WoundClot™ technology is that the material is not soft or extensible. When subject to strain, it tears readily. A positive feature is that it forms an absorbent gel which is highly ionic in nature (sodium chloride) and self-adheres to the skin with minimal pressure (the CMC chemistry).

The knit structure made by Beeken Biomedical under the NuStat brand name is a blend of silica (glass) filaments with bamboo fibers (70/30 blend). Several versions of the knit exist. One is designed as a wrap. Another as a sponge in several sizes. The sponge version is constructed with surface loops like a Terry towel. Highly flexible, thick and absorbent. It is also very soft and stretches. As a 2-layer structure, it can be sewn or laminated. A three-layer structure is possible with a hydrophilic (absorbent) foam core or to an absorbent core containing superabsorbent granules or fibers (SAP or SAF). These 2-layer structures provide mechanical strength, wicking and fluid holding capacity which assist in clot-formation.

A more advanced version involves utilizing a polyurethane (PU) hydrophilic foam as an absorbent core and adhering a surface layer(s) of the knit hemostatic layer, such as the Beeken Biomedical technology, but it could be any of the technologies listed above.

The hydrophilic PU foam absorbs fluids in the 15-20 g/g range. Blood may be somewhat lower. This absorbing action assists the hemostatic knit and overall clot performance is improved. It can also reduce the cost because fewer or lighter layers of the hemostatic knit are required.

A dramatic further improvement is possible if the foam/hemostatic knit is modified and additional hemostatic agents are incorporated into the polymer mix of the foam. Again, generally any of the materials in the bullet-point list above can be incorporated into the foam.

Hydrophilic PU foams are also receptive to post-treatment with liquids which may contain “active” ingredients such as those which promote hemostatic activity. This coating may be 1-side, 2-side or throughout the foam. Printing or patterning the “actives” provides targeted zones of activity or multiple modes of functionality.

Chitosan, zeolites, clays or any ionic material are good candidates for inclusion into the foam. The foam can be loaded with up to 20% by weight in many cases. Other beneficial materials could include antimicrobials.

When this foam is mixed and cast, it generates a gas to self-foam and requires about 3-minutes to solidify and gel. In the last portion of the solidification period, the foam is exceptionally tacky and it makes an excellent adhesive. A material can be laid on one or both sides of the foam and upon chemical completion, these materials are well adhered. A material may also be imbedded into the foam at this stage. The overall structure is soft, flexible, stretchy and highly absorbent. A superb hemostatic material.

A barrier film may be applied to one side which prevents passage of dirt, bacteria or viruses. This approach is well suited to wound dressings or all types.

The foam can be 1-6 mm in thickness, with 1.5-2.0 mm being standard.

The ideal single-layer structure would be constructed via knitting in which fibers or filaments with hemostatic properties compose the majority the interior surface in a Terry or loop configuration while the majority of the exterior surface is non-hemostatic and contains Spandex.

The ideal multilayer structure would be 3-layer consisting of Terry loops on a knit hemostatic surface (Beeken Biomedical type)/a core of hydrophilic foam/the knit outer layer containing Spandex. In summary, the warp knit fabric usually contains at least one of the following: integrated hemostatic agents, silicone or other release agents, and/or an integrated absorbent or wicking feature

It may also include a thermal retention layer or technology for reflection of infrared energy for warmth.

One embodiment of the warp knit fabric is constructed of nylon and spandex (˜87/13) filament yarns with a high density of stitches (80 stitches/inch) and 30 wale/inch yielding 60 filament ends/inch.

Another embodiment is a circular knit fabric with a low density 8-20 stiches/inch.

The cap may be sized to optionally accommodate and hold cold and/or hot packs placed about the patient's head.

The head trauma bandage cap may include antimicrobials placed in or on any of the fibers or filaments. In one embodiment, the antimicrobials are selected from the group comprising silver-ion releasing antimicrobials, quaternary amines and oxidizers, silquats, iodine, chlorine, or chlorhexidine gluconate (CHG). A preferred silver-ion releasing antimicrobial is >10 ppm of elemental silver embedded in a polyurethane polymer blend.

The flexible cap may be color-coded to indicate severity of a patient's injuries in, an emergency trauma triage setting.

The head trauma bandage may be tightened using a conventional bandage or wrapping system, or include a chin strap or other attachment system to aid in securing the head trauma bandage cap on the patient. The chin strap ends are removably attached to the cap via adjustment attachment means, such as corresponding hook and loop strips, which attach to strips placed on the cap exterior. The chin strap may also contain hemostatic or antimicrobial functionality.

The head trauma bandage may be constructed for single usage with removal and repositioning capabilities as a low COF for easy placement. It provides gentle and uniform stretch/compression in the X and Y directions, and can be sterilized without losing stretch/compression properties. It may have a color coded exterior in multiple colors. including white, off-white, or high visibility red, orange, green yellow, etc. with an interior colored white or off-white. Usually continuous filament construction with fine filaments, fine-gauge wales and high stitch density is utilized to minimize cell ingrowth!attachment as is the case with some fibers. >6.0 oz./yd. weight fabric is also preferred.

The head trauma bandage may have a multi-colored logo applied with company name and product information is thermally applies and stretches. An information tag (Made in the USA, size, etc.) may also be included.

The head trauma bandage may be individually packaged, or packaged as a kit with cold packs and absorbent bandages and/or hemostatic agents.

The invention provides the following distinctive features:

Warp, circular or 3D knit which provides conformity and stretch.

Certain woven and nonwoven manufacturing methods may provide the layering, functionality and positioning of hemostatic fibers/filaments with sufficient stretch and recovery to be effective in this bandage application.

The interior layer is made of a fibrous knit structure

Filaments or fibers, blends of filaments or fibers, coated or treated filaments or fibers comprise the hemostatic functionality of the interior layer. These filaments or fibers also provide stretch in the inner layer in conjunction with stretch in the outer layer.

These same fibers may be adhered to the surface of an absorbent medical grade foam, thus creating a hemostatic absorbent medical grade foam.

This head bandage may be sterilized via conventional means although this treatment is optional.

From a field perspective the distinctive features of the stretch bandage are:
Easy, one-person application in less than one minute
Shorter fire and ambulance on-scene time, and faster patient delivery to Emergency Room.
Cap is designed to apply uniform gentle pressure to control bleeding
Cap holds dressings and cold packs in position
The Hemostatic version can reduce or eliminate the need to apply a separate dressing
Can be self-administered
Does not compromise cervical spine immobilization
Will not slip or fall off
Caregiver can lift cap for observation without unwrapping/rewrapping
May provide thermal management
Antibacterial Materials may help prevent infection
Can be utilized in the First Responder, Emergency Room and Post-Surgery settings
Is useful in Tactical Combat, Multi-Casualty and Triage situations

This head bandage may be sterilized via conventional means although this treatment is optional.

Thus, the invention provides a stretch emergency trauma bandage beanie capable of holding wound dressings and/or hot or cold packs, which is placed on the cranium to cover the crown, forehead, ears, back of the head, sides of the head around the ears, and the temples of an injured patient with minimal movement of the neck and spine. It is of simple construction, and is quickly applied in the field.

### BRIEF DESCRIPTION OF THE SEVERAL VIEWS OF THE DRAWINGS

In order that the manner in which the above-recited and other features and advantages of the invention are obtained will be readily understood, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 is a side view of one embodiment of the head trauma bandage cap with liftable edges placed over the ear hole;

FIG. 2 is a side view of the embodiment of FIG. 1 with liftable edges raised above the ear hole;

FIG. 3 is a top front view of the embodiment of FIG. 1;

FIG. 4 is top back view of the embodiment of FIG. 1;

FIG. 5 is a single knit structure interior layer.

FIG. 6 is a 2-layer structure with two knits.

FIG. 7 is a 3-layer structure including a hydrophilic foam or absorbent core.

### DETAILED DESCRIPTION OF THE INVENTION

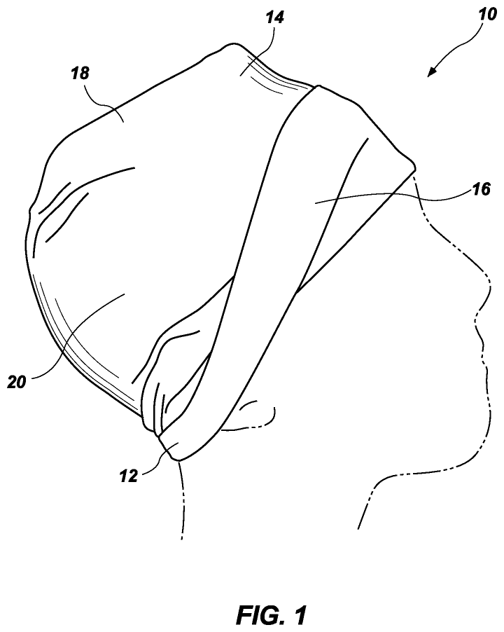

The presently preferred embodiments of the present invention will be best understood by reference to the drawings, wherein like parts are designated by like numerals throughout. It will be readily understood that the components of the present invention, as generally described and illustrated in the figures herein, could be arranged and designed in a wide variety of different configurations. Thus, the following more detailed description of the embodiments of the FIGS. 1-7 of the present invention, is not intended to limit the scope of the invention, as claimed, but is merely representative of presently preferred embodiments of the invention.

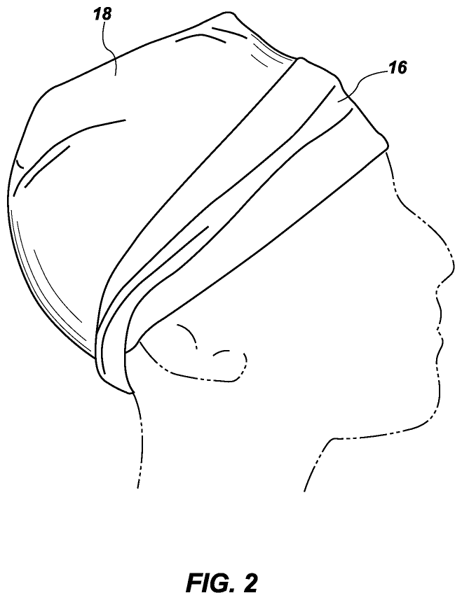

FIG. 1 is a side view of one embodiment of the stretchable head trauma bandage cap 10 with roll able, lift able edges 12 placed over the ear hole. The head trauma bandage cap 10 has two or more layers 14, 16 of a stretchable warp knit fabric cut and sewn to form a form-fitting head bandage shape. The exterior weather resistant or waterproof exterior layer 14 has periphery edges 12, top 18 and sides 20 sized to fit about and cover a forehead/crown, sides and back of a head of a patient with a head trauma. The liftable edges 12 may be rolled up to expose a patient's ears as shown in FIG. 2 to enable caregivers to observe any fluid discharge from the ears. The tightness of the rolled edges 12 can be varied to provide varying head bandage pressure to a head wound.

FIG. 3 is a top front view of the embodiment of FIG. 1;

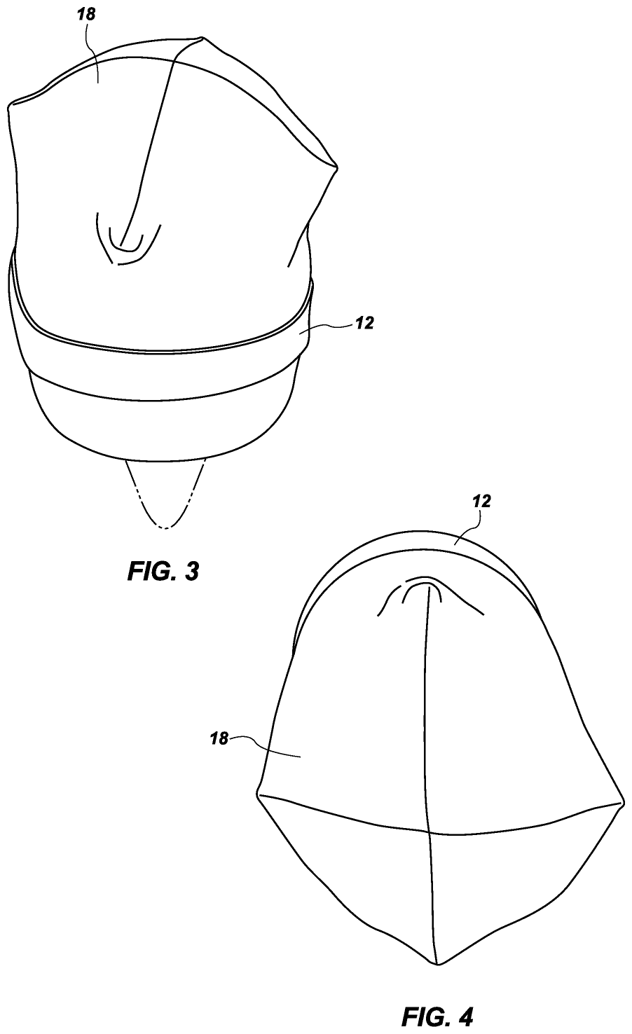

FIG. 4 is top back view of the embodiment of FIG. 1;

The interior layer 16 is made of a sterile superabsorbent wicking polymer non-adherent wound contact-surface, with enough stretch when placed on a patient to apply minimal pressure to a patient's head to control bleeding without aggravating intracranial pressure and can be hydrated and chilled or frozen to prove an extended duration cooling device.

The two layers 14, 16 of the head trauma bandage cap 10 are cut and sewn with seams positioned so no edges are against the patient's skin when applied. The warp knit fabric of the layers 14, 16 may contain at least one of the following: integrated hemostatic agents, silicone or other release agents, and/or an integrated absorbent.

The head trauma bandage cap 10 may include a thermal retention layer or technology for reflection of infrared energy for warmth.

The head trauma bandage cap 10 warp knit fabric is constructed of nylon and spandex (˜87/13) filament yarms. It is sized to accommodate and hold wound dressings and/or cold and/or hot packs placed about the patient's head, and may include antimicrobials placed in the absorbent sterile non-adherent wound-contact surface absorbent layer 16, or in a waterproof-breathable outer layer 14 to reduce bacterial populations and infections. The antimicrobials are selected from the group comprising silver-ion releasing antimicrobials, quaternary amines and oxidizers, silquats, iodine, chlorine, or chlorhexidine gluconate (CHG). The silver-ion releasing antimicrobial is >30 ppm of elemental silver applied as a fiber finish.

The head trauma bandage cap 10 may be color-coded to indicate severity of a patient's injuries in an emergency trauma triage setting.

FIG. 5 is a single knit structure which forms an interior layer 14 with a majority of hemostatic fibers in a Terry or loop knit and an exterior layer 16, which is primarily non hemostatic fibers/filaments.

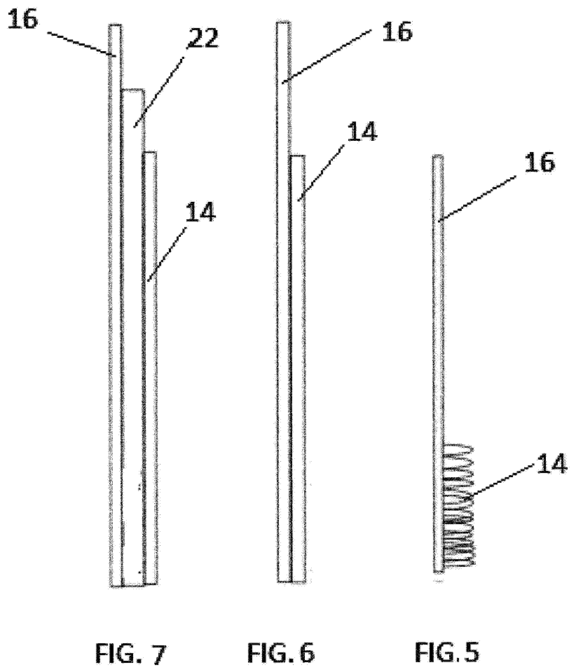

FIG. 6 is a 2-layer structure with two separate knit layers 14, 16. There is a Hemostatic loop knit interior layer 14 and a nylon/spandex knit exterior layer 16 on the outside.

FIG. 7 is a 3-layer structure in which a hydrophilic foam or absorbent core 22 is inserted between the two knit layers 14, 16 of FIG. 6.

The present invention may be embodied in other specific forms without departing from its structures, methods, or other essential characteristics as broadly described herein and claimed hereinafter. The described embodiments are to be considered in all respects only as illustrative, and not restrictive.

We claim:

1. A head trauma stretch bandage cap comprising:
a flexible cap made of two or more layers of a stretchable warp knit fabric cut and sewn to form a form-fitting head bandage skull cap shape with
i. an exterior weather resistant or waterproof exterior with rollable periphery edges, top and sides sized to fit about and cover a forehead/crown, ears, sides and back of a head of a patient with a head trauma; the periphery edges rollable to provide varying tightness, and liftable edges to expose a patient's ears to enable caregivers to observe any fluid discharge from the ears, and
ii. an interior made of a sterile absorbent polymer non-adherent wound contact-surface, with enough stretch when placed on a patient to apply minimal pressure to a patient's head to control bleeding without aggravating intracranial pressure, which can be hydrated and chilled or frozen to prove an extended duration cooling device.

2. A head trauma stretch bandage cap according to claim 1, wherein the two or more layers of stretchable warp knit fabric are cut and sewn with seams positioned so no edges are against the patient's skin when applied.

3. A head trauma stretch bandage cap according to claim 1, wherein the warp knit fabric contains at least one of the following: integrated hemostatic agents, silicone or other release agents, and/or an integrated absorbent.

4. A head trauma stretch bandage cap according to claim 1, including a thermal retention layer or technology for reflection of infrared energy affixed to the interior for warmth.

5. A head trauma stretch bandage cap according to claim 1, wherein at least a portion of the warp knit fabric is constructed of nylon and spandex (˜87/13) filament yarns.

6. A head trauma stretch bandage according to claim 1, wherein the cap is sized to accommodate and hold wound dressings and/or cold and/or hot packs placed about a patient's head.

7. A head trauma stretch bandage cap according to claim 1, including antimicrobials placed in the sterile absorbent polymer non-adherent wound-contact surface, or in an absorbent layer, or in the waterproof exterior to reduce bacterial populations and infections.

8. A head trauma stretch bandage cap according to claim 7, wherein the antimicrobials are selected from the group comprising silver-ion releasing antimicrobials, quaternary amines and oxidizers, silquats, iodine, chlorine, or chlorhexidine gluconate (CHG).

9. A head trauma stretch bandage cap according to claim 8, wherein the silver-ion releasing antimicrobial is >10 ppm of elemental silver applied as a fiber finish.

10. A head trauma stretch bandage cap according to claim 1, wherein the flexible cap is color-coded to indicate severity of a patient's injuries in an emergency trauma triage setting.

11. A head trauma stretch bandage cap according to claim 1, including a strapping or bandage system placed about the flexible cap to selectively increase pressure applied to a patient's head to further control bleeding.

12. A head trauma stretch bandage cap according to claim 1, wherein the cap is a single knit structure in which the interior layer has a majority of hemostatic fibers in a Terry or loop knit and the exterior is primarily constructed of non-hemostatic fibers/filaments.

13. A head trauma stretch bandage cap according to claim 1, wherein the cap is a 2-layer structure with a hemostatic loop knit interior layer and a nylon/spandex knit exterior layer.

14. A head trauma stretch bandage according to claim 13, wherein the cap is a 3-layer structure in which a hydrophilic foam or absorbent core is inserted between the interior layer and the exterior layer.

15. A head trauma stretch bandage according to claim 1, wherein the cap is fabricated using a woven or nonwoven fabric manufacturing process.
